(19) United States
(12) Patent Application Publication
(10) Pub. No.: US 2005/0238583 A1
(43) Pub. Date: Oct. 27, 2005

(54) Contrast agent specifically suitable for X-ray computed tomography

(76) Inventors: Walter Marzendorfer, Erlangen (DE); Bernd Ohnesorge, Erlangen (DE); Stefan Schaller, Furth (DE)

Correspondence Address: SCHIFF HARDIN, LLP;PATENT DEPARTMENT, 6600 SEARS TOWER, CHICAGO, IL, 60606-6473, US

(21) Appl. No.: 11/072,951

(22) Filed: Mar. 3, 2005

(30) Foreign Application Priority Data
Mar. 3, 2004 (DE) 10 2004 010 387.9

(51) Int. Cl. A61K 49/04

(52) U.S. Cl. 424/9.42

(57) ABSTRACT

A contrast agent for medical imaging has a component in the form of a chemical compound of gadolinium that is effective for x-ray computed tomography, that has at least one additive that prevents use of the contrast agent in magnetic resonance tomography.

### BACKGROUND OF THE INVENTION

1. Field of the Invention

The present invention concerns a contrast agent with a component, in the form of a chemical compound of gadolinium, effective for x-ray computed tomography.

2. Description of the Prior Art

Contrast agents are used to increase contrast in an acquired exposure in imaging medical diagnostic methods such as, for example, x-ray computed tomography and magnetic resonance tomography. In x-ray computed tomography, widely used contrast agents contain bonded iodine as an active substance. This substance, however, is tolerable only to a limited degree in patients with renal insufficiency (renal failure) and can lead to side effects.

The suitability of gadolinium as a contrasting active substance for x-ray computed tomography has been proven in various studies, by the selection of a chemical compound of gadolinium as a contrasting active substance, patient incompatibility due to iodine-containing contrast agent is avoided,

A contrast agent of this type is known from German Utility Model 298 00 160,

A contrast agent for use in imaging methods that contains super-paramagnetic particles, in particular for use in magnetic resonance tomography and in x-ray representation, is specified in European Application 1 378 239. The super-paramagnetic particles exhibit an absorption behavior for x-rays that is different from that of the body's own bone tissue, which leads to a contrast improvement. An active substance is additionally applied to the particles, for which a diphosphine acid is preferably used. Due to the diphosphine acid the contrast agent preferably accumulates in the bone tissue, which is why it is particularly suitable for examination of bone metastases. An intensification of the measurement signals is achieved by the super-paramagnetic particles for imaging by means of magnetic resonance.

A contrast agent for magnetic resonance tomography that contains at least one ferromagnetic and/or super-paramagnetic component and at least one paramagnetic component is specified in European Application 0 998 864. Iron or iron oxide is suggested as ferromagnetic or super-paramagnetic components. Gadolinium is used as a paramagnetic component. By the selection of special concentrations of the components, a significantly lower total concentration is required in comparison to contrast agents with only one of the components. The specified contrast agent is particularly suitable for use in virtual endoscopy of cavities [voids].

The use of gadolinium-based contrast agents is typical in magnetic resonance tomography because the paramagnetic properties of gadolinium cause an increase of the magnetic resonance signal in T1-weighted exposures. Given optimal concentration of the gadolinium compound, an intensification of the magnetic resonance signal results, which leads to an improved contrast in the acquired exposures. This concentration of the gadolinium compound in the contrast agent, which is optimal for magnetic resonance tomography, is clearly much too low to also enable a use of the same, low-concentration contrast agent in x-ray computed tomography. A high concentration of the gadolinium compound sufficient for use in x-ray computed tomography leads to high costs for the contrast agent and is therefore uneconomical. A high-concentration contrast agent produced especially for x-ray computed tomography would therefore have to be offered at a price comparable to that of the low-concentration contrast agent so an operator of an x-ray computed tomography apparatus would consider it likewise cost-effective. A problem exists for the contrast agent producer is the risk that an operator of a magnetic resonance tomography apparatus will use the high-concentration contrast agent in diluted form, and revenues from the sale of the low-concentration contrast agent thus will be diminished for the contrast agent producer. It is similarly desirable that use of the high-concentration contrast agent in magnetic resonance tomography be prevented.

### SUMMARY OF THE INVENTION

An object of the present invention is to provide a contrast agent for x-ray computed tomography with an effective component in the form of a chemical compound of gadolinium, such that use of the contrast agent in examinations in magnetic resonance tomography is prevented.

The above object is achieved in accordance with the present invention by a contrast having a component in the form of a chemical compound of gadolinium that is effective for x-ray computed tomography, and at least one additive that prevents use of the contrast agent for magnetic resonance tomography.

The use of such an additive changes the exposure acquired by magnetic resonance tomography so severely that it is unusable for a finding.

### DESCRIPTION OF THE PREFERRED EMBODIMENTS

An advantageous additive Is composed of a Chemical compound with super-paramagnetic properties. The regions of T₁-weighted magnetic resonance exposures that would otherwise be shown light are thereby significantly darker and the exposure is thereby unusable for diagnostic purposes.

An exemplary embodiment is the selection of an additive that contains super-paramagnetic Fe₂O₃ particles, Due to the magnetic properties of these particles, an acceleration of the transversal nuclear spin relaxation results, which leads to a significant reduction of the signal in magnetic resonance tomography. Thus regions are dark in the acquired exposure that would be light given use of the low-concentration, gadolinium-based contrast agent. Magnetic resonance tomography exposures acquired using the contrast agent charged with the Fe₂O₃ particles are thus unusable for a diagnostic. The use in x-ray computed tomography of the contrast agent charged with the Fe₂O₃ particles is non-critical, since no significant change of the x-ray absorption behavior occurs due to the Fe₂O₃ particles. Only the gadolinium compound is decisive in that context.

Although modifications and changes may be suggested by those skilled in the art, it is the intention of the inventors to embody within the patent warranted hereon all changes and modifications as reasonably and properly come within the scope of their contribution to the art.

1. A contrast agent comprising a chemical compound of gadolinium that is effective for x-ray computed tomography, and at least one additive that prevents use of the contrast agent in magnetic resonance tomography.

2. A contrast agent as claimed in claim 1 wherein said additive contains a chemical compound having super-paramagnetic properties.

3. A contrast agent as claimed in claim 2 wherein said additive contains super-paramagnetic Fe₂O₃.
